SEQUENCE LISTING (25 sequences)

SEQ ID NO: 1 - DNA, 1263 bp, Homo Sapiens
atgctgcccgggctcaggcgcctgctgcaagctcccgcctcggcctgcctcctgctgatgctcctggccctgcccctggcggcccccagctgccccatgctctgcacctgctactcatccccgcccaccgtgagctgccaggccaacaacttctcctctgtgccgctgtccctgccacccagcactcagcgactcttcctgcagaacaacctcatccgcacgctgcggccaggcacctttgggtccaacctgctcaccctgtggctcttctccaacaacctctccaccatctacccgggcactttccgccacttgcaagccctggaggagctggacctcggtgacaaccggcacctgcgctcgctggagcccgacaccttccagggcctggagcggctgcagtcgctgcatttgtaccgctgccagctcagcagcctgcccggcaacatcttccgaggcctggtcagcctgcagtacctctacctccaggagaacagcctgctccacctacaggatgacttgttcgcggacctggccaacctgagccacctcttcctccacgggaaccgcctgcggctgctcacagagcacgtgtttcgcggcctgggcagcctggaccggctgctgctgcacgggaaccggctgcagggcgtgcaccgcgcggccttccgcggcctcagccgcctcaccatcctctacctgttcaacaacagcctggcctcgctgcccggcgaggcgctcgccgacctgccctcgctcgagttcctgcggctcaacgctaacccctgggcgtgcgactgccgcgcgcggccgctctgggcctggttccagcgcgcgcgcgtgtccagctccgacgtgacctgcgccacccccccggagcgccagggccgagacctgcgcgcgctccgcgaggccgacttccaggcgtgtccgcccgcggcacccacgcggccgggcagccgcgcccgcggcaacagctcctccaaccacctgtacggggtggccgaggccggggcgcccccagccgatccctccaccctctaccgagatctgcctgccgaagactcgcgggggcgccagggcggggacgcgcctactgaggacgactactgggggggctacgggggtgaggaccagcgaggggagcagatgtgccccggcgctgcctgccaggcgcccccggactcccgaggccctgcgctctcggccgggctccccagccctctgctttgcctcctgctcctggtgccccaccacctctga

SEQ ID NO: 2 - protein, 420 aa, Homo Sapiens
MLPGLRRLLQAPASACLLLMLLALPLAAPSCPMLCTCYSSPPTVSCQANNFSSVPLSLPPSTQRLFLQNNLIRTLRPGTFGSNLLTLWLFSNNLSTIYPGTFRHLQALEELDLGDNRHLRSLEPDTFQGLERLQSLHLYRCQLSSLPGNIFRGLVSLQYLYLQENSLLHLQDDLFADLANLSHLFLHGNRLRLLTEHVFRGLGSLDRLLLHGNRLQGVHRAAFRGLSRLTILYLFNNSLASLPGEALADLPSLEFLRLNANPWACDCRARPLWAWFQRARVSSSDVTCATPPERQGRDLRALREADFQACPPAAPTRPGSRARGNSSSNHLYGVAEAGAPPADPSTLYRDLPAEDSRGRQGGDAPTEDDYWGGYGGEDQRGEQMCPGAACQAPPDSRGPALSAGLPSPLLCLLLLVPHHL

SEQ ID NO: 3 - DNA, 23 bp, Artificial Sequence
  Specific primer GSP1
gtggttggaggaggcctggaagt

SEQ ID NO: 4 - DNA, 22 bp, Artificial Sequence
  Specific primer nGSP1
ggccaggctgttgttgaacagg

SEQ ID NO: 5 - DNA, 23 bp, Artificial Sequence
  Specific primer nGSP2
ggtcagcctgcagtacctctacc

SEQ ID NO: 6 - DNA, 23 bp, Artificial Sequence
  Specific primer NgRH1-3
agtcagaggtggtggggcaccag

SEQ ID NO: 7 - DNA, 22 bp, Artificial Sequence
  Specific primer nGRH1-5
agatgctgcccgggctcaggcg

SEQ ID NO: 8 - DNA, 20 bp, Artificial Sequence
  Synthetic primer
tgaatctggaccccgggagg

SEQ ID NO: 9 - DNA, 23 bp, Artificial Sequence
  Synthetic primer
tcctcagcggagagataccacca

SEQ ID NO: 10 - DNA, 60 bp, Artificial Sequence
  Synthetic oligonucleotide
ccggtaagcctatccctaaccctctcctcggtctcgattctacgtctagatatcctcgag

SEQ ID NO: 11 - DNA, 63 bp, Artificial Sequence
  Synthetic oligonucleotide
gagctcctatagatctgcatcttagctctggctcctctcccaatccctatccgaatggcccga

SEQ ID NO: 12 - DNA, 42 bp, Artificial Sequence
  Synthetic forward primer
gcagcatctagaccaggtgcctgcgtatgctacaatgagccc

SEQ ID NO: 13 - DNA, 42 bp, Artificial Sequence
  Synthetic reverse primer
gcagcactcgagtcagcagggcccaagcactgtccacagcac

SEQ ID NO: 14 - DNA, 53 bp, Artificial Sequence
  Synthetic oligonucleotide
ccgattacaaggatgacgacgataagtctagacagtgcgatatcaatgaattc

SEQ ID NO: 15 - DNA, 56 bp, Artificial Sequence
  Synthetic oligonucleotide
cttaagtaactatagcgtgacagatctgaatagcagcagtaggaacattagcccga

SEQ ID NO: 16 - DNA, 45 bp, Artificial Sequence
  Synthetic forward primer
aattgcgcatctagagcccccagctgccccatgctctgcacctgc

SEQ ID NO: 17 - DNA, 45 bp, Artificial Sequence
  Synthetic reverse primer
aattgcgcagaattctcagaggtggtggggcaccagcagcaggag

SEQ ID NO: 18 - protein, 16 aa, Artificial Sequence
  Synthetic peptide
EQLDLSDNAQLRSVDP

SEQ ID NO: 19 - protein, 17 aa, Artificial Sequence
  Synthetic peptide
EVPCSLPQRLAGRDLKR

SEQ ID NO: 20 - protein, 16 aa, Artificial Sequence
  Synthetic peptide
GPRRRPGCSRKNRTRS

SEQ ID NO: 21 - protein, 16 aa, Artificial Sequence
  Synthetic peptide
CPPAAPTRPGSRARGN

SEQ ID NO: 22 - protein, 16 aa, Artificial Sequence
  Synthetic peptide
DLPAEDSRGRQGGDAP

SEQ ID NO: 23 - protein, 15 aa, Artificial Sequence
  Synthetic peptide
TEDDYWGGYGGEDQR

SEQ ID NO: 24 - DNA, 1263 bp, Rattus norvegicus
atgctgcccgggctccggcgcctgctgcaaggtcctgcctcagcctgcctcctgctgacactcctggccctccctcctgtgacccccagctgccctatgctctgcacctgctactcctctccgcccacagtgagctgccaggccaacaacttctcctcggtgccgctgtccttgccacccagtacacagcgactcttcttgcagaacaacctcattcgctcactgcggccaggaacttttgggcccaacctgctcaccctgtggctcttctccaacaacctctccaccatctaccctggcaccttccgccatctgcaggccctagaggaactggacctcggtgacaatcggcacctgcgctccctggagcctgacaccttccagggcctggagaggctgcagtcactacatctgtaccggtgccagctcagcagtctgcctggcaacatcttccgaggcctggtcagcctacagtacctctacctccaggagaacagcctgctccacctacaggatgacttgttcgccgacctggccaacctgagccaccttttcctccacgggaaccgcctgcggctgctcacggagcacgtgttccgcggcttgggcagcctggaccggctgctgctgcacgggaaccggctgcagggcgtacaccgcgcagccttccacggtctcagccgcctcaccatcctttacctgttcaacaacagcctggcctcgctgccgggagaggcgctggctgacctgccagcgctcgagttcctgcggctcaacgccaacccctgggcgtgcgactgccgcgctcggccgctctgggcttggttccagcgcgcgcgggtgtccagctccgacgtgacctgcgccaccccgcccgagcgccagggccgggacctgcgcacgctgcgcgacaccgatttccaagcgtgcccgccgcccacacccacgcggccgggcagccgcgcccgcggcaacagctcttccaaccacctgtacggcgtggccgaggcgggcgctccccccgcagacccatccacgctctaccgagacctgcccgccgaggactcgcgggggcgtcagggcggggacgcgcccactgaggacgactactgggggggctacggcggcgaggaccagcgaggcgagcagacgtgtcccggggccgcgtgccaggcgcccgcggactcgcgtggccccgtgctctcggccgggctgcgcacccctctgctctgcctcttgctcctggctccccatcacctctga

SEQ ID NO: 25 - protein, 420 aa, Rattus norvegicus
MLPGLRRLLQGPASACLLLTLLALPPVTPSCPMLCTCYSSPPTVSCQANNFSSVPLSLPPSTQRLFLQNNLIRSLRPGTFGPNLLTLWLFSNNLSTIYPGTFRHLQALEELDLGDNRHLRSLEPDTFQGLERLQSLHLYRCQLSSLPGNIFRGLVSLQYLYLQENSLLHLQDDLFADLANLSHLFLHGNRLRLLTEHVFRGLGSLDRLLLHGNRLQGVHRAAFHGLSRLTILYLFNNSLASLPGEALADLPALEFLRLNANPWACDCRARPLWAWFQRARVSSSDVTCATPPERQGRDLRTLRDTDFQACPPPTPTRPGSRARGNSSSNHLYGVAEAGAPPADPSTLYRDLPAEDSRGRQGGDAPTEDDYWGGYGGEDQRGEQTCPGAACQAPADSRGPVLSAGLRTPLLCLLLLAPHHL

(19) United States
(12) Patent Application Publication
(10) Pub. No.: US 2008/0090270 A1
(43) Pub. Date: Apr. 17, 2008

(54) Nogo receptor homologues and their use

(76) Inventors: Carmen Barske, Lorrach (DE); Stefan Frentzel, Lorrach (DE); Andreas Edgar Hein, Lorrach (DE); Klemens Kaupmann, Basel (CH); Bernd Josef Sommer, Ulm (DE)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 11/906,269

(22) Filed: Oct. 1, 2007

Related U.S. Application Data
(63) Continuation of application No. 10/491,810, filed on Apr. 15, 2004, now abandoned.
(60) Provisional application No. 60/337,595, filed on Oct. 22, 2001.

(51) Int. Cl. C12P 21/00; C07H 21/02; C07K 1/00; C07K 16/00; C12N 15/00; C12N 5/00

(52) U.S. Cl. 435/70.1; 435/320.1; 530/387.1; 530/387.3; 530/389.1; 536/23.5

(57) ABSTRACT

This invention relates to gene polypeptides and polynucleotides that encode proteins of the Nogo-66 receptor (NgR) family and are therefore called NgR homologue 1 (NgRH1). The invention further relates to their use in identifying compounds that may be agonists or antagonists that are potentially useful in regeneration and protection of the nervous system, and to production of NgRH1 polypeptides, derivatives, and antibodies.

### FIELD OF THE INVENTION

This invention relates to gene polypeptides and polynucleotides that encode proteins of the Nogo receptor (NgR) family and are therefore called NgR homologues 1 (NgRH1). The invention further relates to their use in identifying compounds that may be agonists or antagonists that are potentially useful in regeneration and protection of the nervous system, and to production of NgRH1 polypeptides, derivatives, and antibodies.

### BACKGROUND OF THE INVENTION

Re-growth of injured neurones in the adult CNS of higher vertebrates is limited due to the presence of inhibitory molecules in myelin or due to the formation of scar tissue. Myelin derived proteins, NogoA and Myelin-Associated Glycoprotein (MAG), have been shown in the past to inhibit neurite outgrowth (Huber and Schwab (2000) Biol. Chem. 381, 407-419). While NogoA is a potent neurite outgrowth inhibitor that restricts the capacity of axonal regeneration in vivo after injury (Bregman et al. (1995) Nature 378, 498-501, Schnell and Schwab (1990) Nature 343, 289-72), MAG was shown to inhibit neurite outgrowth in vitro, depending on the age of the neurones (Mukhopadhyay et al. (1994) Neuron 13, 757-767, DeBellard et al. (1996) Mol. Cell. Neurosci. 7, 89-101).

NogoA is amongst three different variants (NogoA, B and C) the longest splice product of the Nogo gene (Chen et al. (2000) Nature 403, 434-439, GrandPre et al. (2000) Nature 403, 439-444, Prinjha et al. (2000) Nature 403, 383-384) and belongs to the reticulon (RTN) protein family. Neutralising antibodies and the use of different domains of NogoA have delineated two inhibitory domains in the molecule (Chen et al. (2000) Nature 403, 434-439, GrandPre et al. (2000) Nature 403, 439-444, Prinjha et al. (2000) Nature 403, 383-384), one corresponding to the amino terminus of the molecule (amino-NogoA) and the other to an extracellular loop region in the C-terminus, which has been termed Nogo-66 (GrandPre et al. (2000) Nature 403, 439-444).

Myelin-Associated Glycoprotein (MAG) is a member of the immunoglobulin (Ig) family (Lai et al. (1987) Proc. Natl. Acad. Sci. USA 84, 4337-4341, Salzer et al. (1987) J. Cell. Biol. 104, 957-965) and can either promote or inhibit neurite outgrowth depending on the age of the neurones (DeBellard et al. (1996) Mol. Cell. Neurosci. 7, 89-101). Although MAG is also present in Schwann cells of the PNS, it gets non-restrictive to peripheral nerves due to a downregulated after lesioning of peripheral nerves (Martini and Schachner (1988) J. Cell Biol. 106, 1735-1746, Fawcett and Keynes (1990) Annu. Rev. Neurosci. 13, 43-60, Brown et al. (1991) Neuron 6, 359-370).

A receptor, denoted the Nogo-66 receptor (NgR), now appears to play a pivotal role in conveying inhibitory signals from myelin associated proteins to neurones of the CNS. It binds MAG and the oligodendrocyte protein OMgp with similar affinity as the originally discovered ligand Nogo-66 and also mediates inhibition of axonal extensions in vitro and in vivo (Fournier et al. (2001) Nature 409, 341-346, GrandPre and Strittmatter (2002) Nature 417, 547-51, Wang et al. (2002) Nature 417, 941-914, Domeniconi et al. (2002) Neuron 35, 283-290 (published online June 28), Liu et al. (2002) Science June 27 (epub ahead of print). It is specifically expressed in the brain and is regulated during development (Wang et al. (2002) J. Neurosci. 22, 5505-5515). NgR is a member of the proteoglycan/leucine-rich-repeat protein family and is attached to the cell surface by a C-terminal glyosyl-phosphatidyinositol (GPI) anchor. The NgR protein sequence contains eight leucine-rich-repeats (LRR) followed by a leucine-rich-repeat C-terminus (LRRCT). These motifs are found in a functionally and evolutionarily diverse set of proteins, including adhesion molecules and signal-transducing receptors (Kobe and Deisenhofer (1994) TIBS 19, 415-421).

Recently, a NgR antagonist peptide, comprising the N-terminal 40 amino acids of Nogo-66, was shown to induce regeneration in spinal cord injury and also improved functional recovery, providing a potential therapeutic for CNS injuries (GrandPre and Strittmatter (2002) Nature 417, 547-51).

### SUMMARY OF THE INVENTION

In a first aspect, the invention provides an isolated DNA from human origin comprising a nucleotide sequence as set forth in SEQ ID NO: 1 and termed human NgRH1 cDNA. In a further aspect, the invention relates to rat NgRH1 cDNA as set forth in SEQ ID NO: 24. A further aspect the invention relates to rat and/or human type NgRH1 polypeptides.

Such polypeptides include:

(a) an isolated polypeptide encoded by a polynucleotide comprising the sequence of SEQ ID NO: 1 or SEQ ID NO: 24;

(b) an isolated polypeptide comprising a polypeptide sequence having at least 80%, 85%, 90%, 95%, 96%, 97%, 98%, or 99% Identity to the polypeptide sequence of SEQ ID NO: 2 or SEQ ID NO: 25;

(c) an isolated polypeptide comprising the polypeptide sequence of SEQ ID NO: 2 or SEQ ID NO: 25;

(d) an isolated polypeptide having at least 80%, 85%, 90%, 95%, 96%, 97%, 98%, or 99% Identity to the polypeptide sequence of SEQ ID NO: 2 or SEQ ID NO: 25;

(e) the polypeptide sequence of SEQ ID NO: 2 or SEQ ID NO: 25; and

(f) an isolated polypeptide having or comprising a polypeptide, sequence that has an Identity Index of 0.80, 0.85, 0.90, 0.95, 0.96, 0.97, 0.98, or 0.99 compared to the polypeptide sequence of SEQ ID NO: 2 or SEQ ID NO: 25;

(g) fragments and variants of such polypeptides in (a) to (f).

Also provided are nucleic acid sequences comprising at least about 15 bases, preferably at least about 20 bases, more preferably a nucleic acid sequence comprising about 30 contiguous bases of SEQ ID NO: 1 or SEQ ID NO: 24. Also within the scope of the present invention are nucleic acids that are substantially similar to the nucleic acid with the nucleotide sequence as set forth in SEQ ID NO: 1 or SEQ ID NO: 24. In a preferred embodiment, the isolated DNA takes the form of a vector molecule comprising the DNA as set forth in SEQ ID NO: 1 or SEQ ID NO: 24.

In a second aspect, the invention provides an isolated polypeptide with an amino acid sequence as set forth in SEQ ID NO: 2 or SEQ ID NO: 25. Fragments of the isolated polypeptide with an amino acid sequence as set forth in SEQ ID NO: 2 or SEQ ID NO: 25 μl comprise polypeptides comprising from about 5 to 410 amino acids, preferably from about 10 to about 400 amino acids, more preferably from about 20 to about 100 amino acids, and most preferably from about 20 to about 50 amino acids. In accordance with this aspect of the invention there are provided novel polypeptides of human origin as well as biologically, diagnostically or therapeutically useful fragments, variants and derivatives thereof, variants and derivatives of the fragments, and analogs of the foregoing.

In a third aspect the invention provides the use of modulators of NgRH1 as therapeutic agents. Modulators described herein, include but are not limited to agonists, antagonists, suppressors and inducers of NgRH1.

In a further aspect of the invention there are provided nucleotide probes that are useful for detection of mRNA of the NgRH1 and anti-sense polynucleotides that regulate translation of NgRH genes; In another embodiment, double stranded RNAs provided that can regulate the transcription of NgRH1 genes. This includes small interfering RNAs (sIRNAs) according to standard procedures (Zamore et al. (2000) Cell 101, 25-33; Elbashir et al. (2001) Nature 411, 494-498).

Another aspect of the invention provides a process for producing the aforementioned polypeptides, polypeptide fragments, variants and derivatives, fragments of the variants and derivatives, and analogs of the foregoing. In a preferred embodiment of this aspect of the invention there are provided methods for producing the aforementioned human NgRH1 polypeptides comprising culturing host cells having incorporated therein an expression vector containing an exogenously-derived NgRH1-encoding polynucleotide under conditions sufficient for expression of NgRH1 polypeptides in the host and then recovering the expressed polypeptide.

In accordance with another aspect of the invention there are provided products, compositions, processes and methods that utilize the aforementioned polypeptides and polynucleotides for, inter alia, research, biological, clinical and therapeutic purposes.

In certain additional preferred embodiments of this aspect of the invention there are provided an antibody or a fragment thereof which specifically binds to a polypeptide that comprises the amino acid sequence set forth in SEQ ID NO: 2 or SEQ ID NO: 25, i.e., human or rat NgRH1. In certain particularly preferred embodiments in this regard, the antibodies are highly selective for human NgRH1 polypeptides or portions of human NgRH1 polypeptides.

In a further aspect, an antibody or fragment thereof. Is provided that binds to a fragment or portion of the amino add sequence set forth in SEQ ID NO: 2 or SEQ ID NO: 25.

In another aspect, there are provided methods for treatment of diseases, disorders or damage which ultimately result in damage of the nervous system in a subject, where the disease is mediated by or associated with an increase or decrease in NgRH1 gene expression or an increase or decrease in the presence of NgRH1 polypeptide in all major brain regions (except pons), skeletal muscle and liver. Such diseases, disorders or damage include, but are not limited to, central nervous system (CNS) trauma (e.g. spinal cord or brain injuries), infarction, infection, malignancy, exposure to toxic agents, nutritional deficiency, paraneoplastic syndromes, and degenerative nerve diseases (including but not limited to Alzheimer's disease, Parkinson's disease, Huntington's Chorea, multiple sclerosis, amylotrophic lateral sclerosis, and progressive supra-nuclear palsy). The treatment may be achieved by administering compounds that interfere with NgRH1 activity (e.g. antibodies to NgRH1, anti-sense nucleic acids of NgRH1 (siRNAs according to Zamore et al. (2000) Cell 101, 25-33 or Elbashir et al. (2001) Nature 411, 494-498), NgRH1 ribozymes or chemical groups that bind to the active site of NgRH1.

Another aspect is directed to pharmaceutical compositions comprising an antibody that binds to NgRH1 polypeptides or a fragment thereof for the treatment of acute and chronic neurodegenerative diseases (e.g. as mentioned above), trauma and degenerative eye diseases, brain and spinal trauma, stroke, spinal cord injuries.

In yet another aspect, the invention is directed to methods for the identification of molecules that can bind to NgRH1 polypeptides and/or modulate the activity of NgRH1 polypeptides or molecules that can bind to nucleic acid sequences that modulate the transcription or translation of NgRH1 polypeptides. Such methods are disclosed in, e.g., U.S. Pat. No. 6,043,024, incorporated by reference herein in its entirety. Molecules identified by such methods also fall within the scope of the present invention.

In yet another aspect, the invention provides cells which can be propagated in vitro, preferably vertebrate cells, which are capable upon growth in culture of producing a polypeptide that comprises the amino acid sequence set forth in SEQ ID NO: 2 or SEQ ID NO: 25 or fragments thereof, where the cells contain transcriptional control DNA sequences, other than human NgRH1 transcriptional control sequences, where the transcriptional control sequences control transcription of DNA encoding a polypeptide with the amino acid sequence according to SEQ ID NO: 2 or SEQ ID NO: 25 or fragments thereof.

In another aspect, the present invention provides a method for producing NgRH1 polypeptides which comprises culturing a host cell having incorporated therein an expression vector containing an exogenously-derived NgRH1-encoding polynucleotide under conditions sufficient for expression of NgRH1 polypeptides in the host cell, thereby causing the production of an expressed polypeptide, and recovering the expressed polypeptide.

Other objects, features, advantages and aspects of the present invention will become apparent to those of skill from the following description. It should be understood, however, that the following description and the specific examples, while indicating preferred embodiments of the invention, are given by way of illustration only. Various changes and modifications within the spirit and scope of the disclosed invention will become readily apparent to those skilled in the art from reading the following description and from reading the other parts of the present disclosure.

### DETAILED DESCRIPTION OF THE INVENTION

All patent applications, patents and literature references cited herein are hereby incorporated by reference in their entirety.

In practicing the present invention, many conventional techniques in molecular biology, microbiology, and recombinant DNA are used. These techniques are well known and are explained in, for example, Current Protocols in Molecular Biology, Volumes I, II, and III, 1997 (F. M. Ausubel ed.); Sambrook et al., 1989, Molecular Cloning: A Laboratory Manual, Second Edition, Cold Spring Harbor Laboratory Press, Cold Spring Harbor, N.Y.; DNA Cloning: A Practical Approach, Volumes I and II, 1985 (D. N. Glover ed.); Oligonucleotide Synthesis, 1984 (M. L. Gait ed.); Nucleic Acid Hybridization, 1985, (Hames and Higgins); Transcription and Translation, 1984 (Hames and Higgins eds.); Animal Cell Culture, 1986 (R. I. Freshney ed.); Immobilized Cells and Enzymes, 1986 (IRL Press); Perbal, 1984, A Practical Guide to Molecular Cloning; the series, Methods in Enzymology (Academic Press, Inc.); Gene Transfer Vectors for Mammalian Cells, 1987 (J. H. Miller and M. P. Calos eds., Cold Spring Harbor Laboratory); and Methods in Enzymology Vol. 154 and Vol. 155 (Wu and Grossman, and Wu, eds., respectively).

As used herein, “differentially expressed gene” refers to (a) a gene containing at least one of the DNA sequences disclosed herein (e.g., as shown in SEQ ID NO: 1 or SEQ ID NO: 24); (b) any DNA sequence that encodes the amino acid sequence encoded by the DNA sequences disclosed herein (e.g., as shown in SEQ ID NO: 2 or SEQ ID NO: 25); or (c) any DNA sequence that is substantially similar to the coding sequences disclosed herein. For example, the invention provides NgRH1 genes and their encoded proteins of many different species. In specific embodiments, the NgRH1 genes and proteins are from vertebrates, or more particularly, mammals. In a preferred embodiment of the invention, the NgRH1 gene and proteins are from human origin. In its broadest sense, the term “substantially similar”, when used herein with respect to a nucleotide sequence, means a nucleotide sequence corresponding to a reference nucleotide sequence, wherein the corresponding sequence encodes a polypeptide having substantially the same structure and function as the polypeptide encoded by the reference nucleotide sequence, e.g. they are capable of displaying one or more known functional activities (e.g. preventing regeneration of neurons in the spinal cord or brain, conferring to a substrate the property of restricting growth, spreading, and migration of neural cells, and neoplastic cells, inhibiting dorsal root ganglia neurite outgrowth, inducing dorsal root ganglia growth cone collapse, blocking NIH 3T3 cell spreading in vitro, blocking PC12 neurite outgrowth, restricting plasticity) associated with a full-length (wild-type) NgRH1 protein. Desirably the substantially similar nucleotide sequence encodes the polypeptide encoded by the reference nucleotide sequence. The percentage of identity between the substantially similar nucleotide sequence and the reference nucleotide sequence desirably is at least 80%, more desirably at least 85%, preferably at least 90%, more preferably at least 95, 96, 97, 98%, still more preferably at least 99%. Sequence comparisons are carried out using a Smith-Waterman sequence alignment algorithm (see e.g. Waterman, M. S. Introduction to Computational Biology: Maps, sequences and genomes. Chapman & Hall. London: 1995. ISBN 0-412-99391-0). A nucleotide sequence “substantially similar” to reference nucleotide sequence hybridizes to the reference nucleotide sequence in 7% sodium dodecyl sulfate (SDS), 0.5 M NaPO₄, 1 mM EDTA at 50° C. with washing in 2×SSC, 0.1% SDS at 50° C., more desirably in 7% sodium dodecyl sulfate (SDS), 0.5 M NaPO₄, 1 mM EDTA at 50° C. with washing in 1×SSC, 0.1% SDS at 50° C., more desirably still in 7% sodium dodecyl sulfate (SDS), 0.5 M NaPO₄, 1 mM EDTA at 50° C. with washing in 0.5×SSC, 0.1% SDS at 50° C., preferably in 7% sodium dodecyl sulfate (SDS), 0.5 M NaPO₄, 1 mM EDTA at 50° C. with washing in 0.1×SSC, 0.1% SDS at 50° C., more preferably in 7% sodium dodecyl sulfate (SDS), 0.5 M NaPO₄, 1 mM EDTA at 50° C. with washing in 0.1×SSC, 0.1% SDS at 65° C., yet still encodes a functionally equivalent gene product.

Inhibition of the activity mediated by NgRH1 proteins can permit regeneration of neurons in the spinal cord or brain; confer to a substrate the property of permissive growth; the spreading and migration of neural cells and neoplastic cells; allow dorsal root ganglia neurite outgrowth; induce dorsal root ganglia growth cone growth; permit NIH 3T3 cell spreading in vitro; permit PC12 neurite outgrowth and plasticity.

A “host cell,” as used herein, refers to a prokaryotic or eukaryotic cell that contains heterologous DNA that has been introduced into the cell by any means, e.g., electroporation, calcium phosphate precipitation, microinjection, transformation, viral infection, and the like.

“Heterologous” as used herein means “of different natural origin” or represent a non-natural state. For example, if a host cell is transformed with a DNA or gene derived from another organism, particularly from another species, that gene is heterologous with respect to that host cell and also with respect to descendants of the host cell which carry that gene. Similarly, heterologous refers to a nucleotide sequence derived from and inserted into the same natural, original cell type, but which is present in a non-natural state, e.g. a different copy number, or under the control of different regulatory elements.

“Identity” reflects a relationship between two or more polypeptide sequences or two or more polynucleotide sequences, determined by comparing the sequences. In general, Identity refers to an exact nucleotide to nucleotide or amino add to amino add correspondence of the two polynucleotide or two polypeptide sequences, respectively, over the length of the sequences being compared.

“% Identity”—For sequences where there is not an exact correspondence, a “% Identity” may be determined. In general, the two sequences to be compared are aligned to give a maximum correlation between the sequences. This may include inserting “gaps” In either one or both sequences, to enhance the degree of alignment. A % Identity may be determined over the whole length of each of the sequences being compared (so-called global alignment), that is particularly suitable for sequences of the same or very similar length, or over shorter, defined lengths (so-called local alignment), that is more suitable for sequences of unequal length.

“Similarity” Is a further, more sophisticated measure of the relationship between two polypeptide sequences. In general, “similarity” means a comparison between the amino acids of two polypeptide chains, on a residue by residue basis, taking into account not only exact correspondences between pairs of residues, one from each of the sequences being compared (as for identity) but also, where there is not an exact correspondence, whether, on an evolutionary basis, one residue is a likely substitute for the other. This likelihood has an associated “score” from which the “% similarity” of the two sequences can then be determined.

Methods for comparing the identity and similarity of two or more sequences are well known in the art. Thus for instance, programs available in the Wisconsin Sequence Analysis Package, version 9.1 (Devereux J et al, Nucleic Acids Res, 12, 387-395, 1984, available from Genetics Computer Group, Madison, Wis., USA), for example the programs BESTFIT and GAP, may be used to determine the % identity between two polynucleotides and the % identity and the % similarity between two polypeptide sequences. BESTFIT uses the “local homology” algorithm of Smith and Waterman (J Mol Biol, 147,195-197, 1981, Advances in Applied Mathematics, 2, 482-489, 1981) and finds the best single region of similarity between two sequences. BESTFIT is more suited to comparing two polynucleotide or two polypeptide sequences that are dissimilar in length, the program assuming that the shorter sequence represents a portion of the longer. In comparison, GAP aligns two sequences, finding a “maximum similarity”, according to the algorithm of Neddleman and Wunsch (J Mol Biol, 48, 443-453, 1970). GAP is more suited to comparing sequences that are approximately the same length and an alignment is expected over the entire length. Preferably, the parameters “Gap Weight” and “Length Weight” used in each program are 50 and 3, for polynucleotide sequences and 12 and 4 for polypeptide sequences, respectively. Preferably, % identities and similarities are determined when the two sequences being compared are optimally aligned. Other programs for determining identity and/or similarity between sequences are also known in the art, for instance the BLAST family of programs (Altschul S F et al, J Mol Biol, 215, 403-410, 1990, Altschul S F et al, Nucleic Acids Res., 25:389-3402, 1997, available from the National Center for Biotechnology Information (NCBI), Bethesda, Md., USA and accessible through the home page of the NCBI at www.ncbl.nlm.nih.gov) and FASTA (Pearson W R, Methods in Enzymology, 183, 63-99, 1990; Pearson W R and Lipman D J, Proc Nat Aced Sci USA, 85, 2444-2448, 1988, available as part of the Wisconsin Sequence Analysis Package).

Preferably, the BLOSUM62 amino acid substitution matrix (Henikoff S and Henikoff J G, Proc. Nat. Aced Sci. USA, 89, 10915-10919, 1992) is used in polypeptide sequence comparisons including where nucleotide sequences are first translated into amino acid sequences before comparison.

Preferably, the program BESTFIT is used to determine the % identity of a query polynucleotide or a polypeptide sequence with respect to a reference polynucleotide or a polypeptide sequence, the query and the reference sequence being optimally aligned and the parameters of the program set at the default value, as hereinbefore described.

A vector molecule is a nucleic acid molecule into which heterologous nucleic acid may be inserted which can then be introduced into an appropriate host cell. Vectors preferably have one or more origin of replication, and one or more site into which the recombinant DNA can be inserted. Vectors often have convenient means by which cells with vectors can be selected from those without, e.g., they encode drug resistance genes. Common vectors include plasmids, viral genomes, and (primarily in yeast and bacteria) “artificial chromosomes.”

“Plasmids” generally are designated herein by a lower case p preceded and/or followed by capital letters and/or numbers, in accordance with standard naming conventions that are familiar to those of skill in the art. Starting plasmids disclosed herein are either commercially available, publicly available on an unrestricted basis, or can be constructed from available plasmids by routine application of well known, published procedures. Many plasmids and other cloning and expression vectors that can be used in accordance with the present invention are well known and readily available to those of skill in the art. Moreover, those of skill readily may construct any number of other plasmids suitable for use in the invention. The properties, construction and use of such plasmids, as well as other vectors, in the present invention will be readily apparent to those of skill from the present disclosure. The term “isolated” means that the material is removed from its original environment (e.g., the natural environment if it is naturally occurring). For example, a naturally-occurring polynucleotide or polypeptide present in a living animal is not isolated, but the same polynucleotide or polypeptide, separated from some or all of the coexisting materials in the natural system, is isolated, even if subsequently reintroduced into the natural system. Such polynucleotides could be part of a vector and/or such polynucleotides or polypeptides could be part of a composition, and still be isolated in that such vector or composition is not part of its natural environment.

As used herein, the term “transcriptional control sequence” refers to DNA sequences, such as initiator sequences, enhancer sequences, and promoter sequences, which induce, repress, or otherwise control the transcription of protein encoding nucleic acid sequences to which they are operably linked.

The term “polypeptide” is used interchangeably herein with the terms “polypeptides” and “protein(s)”.

As used herein, a “chemical derivative” of a polypeptide of the invention is a polypeptide of the invention that contains additional chemical moieties not normally a part of the molecule. Such moieties may improve the molecule's solubility, absorption, biological half life, etc. The moieties may alternatively decrease the toxicity of the molecule, eliminate or attenuate any undesirable side effect of the molecule, etc. Moieties capable of mediating such effects are disclosed, for example, In Remington's Pharmaceutical Sciences, 16th ed., Mack Publishing Co., Easton, Pa. (1980).

The invention includes nucleic acid molecules, preferably DNA molecules, such as (1) an isolated DNA comprising a nucleotide sequence as set forth in SEQ ID NO: 1 or SEQ ID NO: 24, (2) isolated DNA's that comprise nucleic acid sequences that hybridize under high stringency conditions to the isolated DNA as set forth in SEQ ID NO: 1 or SEQ ID NO: 24, and (3) nucleic acid sequences that hybridize to (1) or (2), above. Such hybridization conditions may be highly stringent or less highly stringent, as described above. In instances wherein the nucleic acid molecules are deoxyoligonucleotides (“oligos”), highly stringent conditions may refer, e.g., to washing in 6×SSC/0.05% sodium pyrophosphate at 37° C. (for 14-base oligos), 48° C. (for 17-base oligos), 55° C. (for 20-base oligos), and 60° C. (for 23-base oligos). Suitable ranges of such stringency conditions for nucleic acids of varying compositions are described e.g. In Krause and Aaronson (1991) Methods in Enzymology, 200:546-556.

These nucleic acid molecules may act as target gene antisense molecules, useful, for example, in target gene regulation and/or as antisense primers in amplification reactions of target gene nucleic acid sequences.

The invention also encompasses (a) vectors that contain any of the foregoing coding sequences and/or their complements (i.e., antisense); (b) expression vectors that contain any of the foregoing coding sequences operatively associated with a regulatory element that directs the expression of the coding sequences; and (c) genetically engineered host cells that contain any of the foregoing coding sequences operatively associated with a regulatory element that directs the expression of the coding sequences in the host cell. As used herein, regulatory elements include but are not limited to inducible and non-Inducible promoters, enhancers, operators and other elements known to those skilled in the art that drive and regulate expression.

The invention includes fragments of any of the nucleic acid sequences disclosed herein. Fragments of the full length NgRH1 gene may be used as a hybridization probe for a cDNA library to isolate the full length gene and to isolate other genes which have a high sequence similarity to the NgRH1 gene or similar biological activity. Probes of this type preferably have at least about 30 bases and may contain, for example, from about 30 to about 50 bases, about 50 to about 100 bases, about 100 to about 200 bases, or more than 200 bases. The probe may also be used to identify a cDNA clone corresponding to a full length transcript and a genomic clone or clones that contain the complete NgRH1 gene including regulatory and promoter regions, exons, and introns. An example of a screen comprises isolating the coding region of the NgRH1 gene by using the known DNA sequence to synthesize an oligonucleotide probe. Labeled oligonucleotides having a sequence complementary to that of the gene of the present invention are used to screen a library of human cDNA, genomic DNA or mRNA to determine which members of the library the probe hybridizes to.

In addition to the gene sequences described above, homologs of such sequences, as may, for example be present in other species, may be identified and may be readily isolated, without undue experimentation, by molecular biological techniques well known in the art. Further, there may exist genes at other genetic loci within the genome that encode proteins which have extensive homology to one or more domains of such gene products. These genes may also be identified via similar techniques.

For example, the isolated differentially expressed gene sequence may be labeled and used to screen a cDNA library constructed from mRNA obtained from the organism of interest. Hybridization conditions will be of a lower stringency when the cDNA library was derived from an organism different from the type of organism from which the labeled sequence was derived. Alternatively, the labeled fragment may be used to screen a genomic library derived from the organism of interest, again, using appropriately stringent conditions. Such low stringency conditions will be well known to those of skill in the art, and will vary predictably depending on the specific organisms from which the library and the labeled sequences are derived.

Further, a previously unknown differentially expressed gene-type sequence may be isolated by performing PCR using two degenerate oligonucleotide primer pools designed on the basis of amino acid sequences within the gene of interest. The template for the reaction may be cDNA obtained by reverse transcription of mRNA prepared from human or non-human cell lines or tissue known or suspected to express a differentially expressed gene allele.

The PCR product may be subcloned and sequenced to ensure that the amplified sequences represent the sequences of a differentially expressed gene-like nucleic add sequence. The PCR fragment may then be used to isolate a full length cDNA clone by a variety of methods. For example, the amplified fragment may be labeled and used to screen a bacteriophage cDNA library. Alternatively, the labeled fragment may be used to screen a genomic library.

PCR technology may also be utilized to isolate full length cDNA sequences. For example, RNA may be isolated, following standard procedures, from an appropriate cellular or tissue source. A reverse transcription reaction may be performed on the RNA using an oligonucleotide primer specific for the most 5′ end of the amplified fragment for the priming of first strand synthesis. The resulting RNA/DNA hybrid may then be “tailed” with guanines using a standard terminal transferase reaction, the hybrid may be digested with RNAase H, and second strand synthesis may then be primed with a poly-C primer. Thus, cDNA sequences upstream of the amplified fragment may easily be isolated.

Preferred polypeptides and polynucleotides of the present invention are expected to have, inter alia, similar biological functions/properties to their homologous polypeptides and polynucleotides. Furthermore, preferred polypeptides and polynucleotides of the present invention have at least one activity of human or rat NgRH1.

A variety of host-expression vector systems may be utilized to express the differentially expressed gene coding sequences of the invention. Such host-expression systems represent vehicles by which the coding sequences of interest may be produced and subsequently purified, but also represent cells which may, when transformed or transfected with the appropriate nucleotide coding sequences, exhibit the differentially expressed gene protein of the invention in situ. These include but are not limited to microorganisms such as bacteria (e.g., E. coli, B. subtilis) transformed with recombinant bacteriophage DNA, plasmid DNA or cosmid DNA expression vectors containing differentially expressed gene protein coding sequences; yeast (e.g. Saccharomyces, Pichia) transformed with recombinant yeast expression vectors containing the differentially expressed gene protein coding sequences; insect cell systems infected with recombinant virus expression vectors (e.g., baculovirus) containing the differentially expressed gene protein coding sequences; plant cell systems infected with recombinant virus expression vectors (e.g., cauliflower mosaic virus, CaMV; tobacco mosaic virus, TMV) or transformed with recombinant plasmid transformation vectors (e.g. Ti plasmid) containing differentially expressed gene protein coding sequences; or mammalian cell systems (e.g. COS, CHO, BHK, 293, 3T3) harboring recombinant expression constructs containing promoters derived from the genome of mammalian cells (e.g., metallothioneine promoter) or from mammalian viruses (e.g., the adenovirus late promoter; the vaccinia virus 7.5K promoter).

In bacterial systems, a number of expression vectors may be advantageously selected depending upon the use intended for the differentially expressed gene protein being expressed. For example, when a large quantity of such a protein is to be produced, for the generation of antibodies or to screen peptide libraries, for example, vectors which direct the expression of high levels of fusion protein products that are readily purified may be desirable. Such vectors include, but are not limited, to the E. coli expression vector pUR278 (Ruther et al., 1983, EMBO J. 2:1791), In which the differentially expressed gene protein coding sequence may be ligated individually into the vector in frame with the lac Z coding region so that a fusion protein is produced; pIN vectors (e.g. Inouye & Inouye, 1985, Nucleic Acids Res. 13:3101-3109); and the like. PGEX vectors may also be used to express foreign polypeptides as fusion proteins with glutathione S-transferase (GST). In general, such fusion proteins are soluble and can easily be purified from lysed cells by adsorption to glutathione-agarose beads followed by elution in the presence of free glutathione. The PGEX vectors are designed to include thrombin or factor Xa protease cleavage sites so that the cloned target gene protein can be released from the GST moiety.

Promoter regions can be selected from any desired gene using vectors that contain a reporter transcription unit lacking a promoter region, such as a chloramphenicol acetyl transferase (“cat”) transcription unit, downstream of restriction site or sites for introducing a candidate promoter fragment; i.e., a fragment that may contain a promoter. As is well known, introduction into the vector of a promoter-containing fragment at the restriction site upstream of the cat gene engenders production of CAT activity, which can be detected by standard CAT assays. Vectors suitable to this end are well known and readily available. Two such vectors are pKK232-8 and pCM7. Thus, promoters for expression of polynucleotides of the present invention include not only well known and readily available promoters, but also promoters that readily may be obtained by the foregoing technique, using a reporter gene.

Among known bacterial promoters suitable for expression of polynucleotides and polypeptides in accordance with the present invention are the E. coli lacI and lacZ promoters, the T3 and T7 promoters, the T5 tac promoter, the lambda PR, PL promoters and the trp promoter. Among known eukaryotic promoters suitable in this regard are the CMV immediate early promoter, the HSV thymidine kinase promoter, the early and late SV40 promoters, the promoters of retroviral LTRs, such as those of the Rous sarcoma virus (“RSV”), and metallothionein promoters, such as the mouse metallothionein-I promoter.

In an insect system, Autographa californica nuclear polyhedrosis virus (AcNPV) is one of several insect systems that can be used as a vector to express foreign genes. The virus grows in Spodoptera frugiperda cells. The differentially expressed gene coding sequence may be cloned individually into non-essential regions (for example the polyhedrin gene) of the virus and placed under control of an AcNPV promoter (for example the polyhedrin promoter). Successful insertion of differentially expressed gene coding sequence will result in inactivation of the polyhedrin gene and production of non-occluded recombinant virus (i.e., virus lacking the proteinaceous coat coded for by the polyhedrin gene). These recombinant viruses are then used to infect Spodoptera frugiperda cells in which the inserted gene is expressed. (E.g., see Smith et al., 1983, J. Virol. 46: 584; Smith, U.S. Pat. No. 4,215,051). In mammalian host cells, a number of viral-based expression systems may be utilized. In cases where an adenovirus is used as an expression vector, the differentially expressed gene coding sequence of interest may be ligated to an adenovirus transcription/translation control complex, e.g., the late promoter and tripartite leader sequence. This chimeric gene may then be inserted in the adenovirus genome by in vitro or in vivo recombination. Insertion in a non-essential region of the viral genome (e.g., region E1 or E3) will result in a recombinant virus that is viable and capable of expressing differentially expressed gene protein in infected hosts. (E.g., See Logan & Shenk, 1984, Proc. Natl. Aced. Sci. USA 81:3655-3659). Specific initiation signals may also be required for efficient translation of inserted differentially expressed gene coding sequences. These signals include the ATG initiation codon and adjacent sequences. In cases where an entire differentially expressed gene, including its own initiation codon and adjacent sequences, is inserted into the appropriate expression vector, no additional translational control signals may be needed. However, in cases where only a portion of the differentially expressed gene coding sequence is inserted, exogenous translational control signals, including, perhaps, the ATG initiation codon, must be provided. Furthermore, the initiation codon must be in phase with the reading frame of the desired coding sequence to ensure translation of the entire insert. These exogenous translational control signals and initiation codons can be of a variety of origins, both natural and synthetic. The efficiency of expression may be enhanced by the inclusion of appropriate transcription enhancer elements, transcription terminators, etc. (see Bittner et al., 1987, Methods in Enzymol. 153:516-544).

Selection of appropriate vectors and promoters for expression in a host cell is a well known procedure and the requisite techniques for expression vector construction, introduction of the vector into the host and expression in the host per se are routine skills in the art.

Generally, recombinant expression vectors will include origins of replication, a promoter derived from a highly-expressed gene to direct transcription of a downstream structural sequence, and a selectable marker to permit isolation of vector containing cells after exposure to the vector.

In addition, a host cell strain may be chosen which modulates the expression of the inserted sequences, or modifies and processes the gene product in the specific fashion desired. Such modifications (e.g., glycosylation) and processing (e.g., cleavage) of protein products may be important for the function of the protein. Different host cells have characteristic and specific mechanisms for the post-translational processing and modification of proteins. Appropriate cell lines or host systems can be chosen to ensure the correct modification and processing of the foreign protein expressed. To this end, eukaryotic host cells which possess the cellular machinery for proper processing of the primary transcript, glycosylation, and phosphorylation of the gene product may be used. Such mammalian host cells include but are not limited to CHO, VERO, BHK, HeLa, COS, MDCK, 293, 3T3, WI38, etc.

For long-term, high-yield production of recombinant proteins, stable expression is preferred. For example, cell lines which stably express the differentially expressed gene protein may be engineered. Rather than using expression vectors which contain viral origins of replication, host cells can be transformed with DNA controlled by appropriate expression control elements (e.g., promoter, enhancer, sequences, transcription terminators, polyadenylation sites, etc.), and a selectable marker. Following the introduction of the foreign DNA, engineered cells may be allowed to grow for 1-2 days in an enriched media, and then are switched to a selective media. The selectable marker in the recombinant plasmid confers resistance to the selection and allows cells to stably integrate the plasmid into their chromosomes and grow to form foci which in turn can be cloned and expanded into cell lines. This method may advantageously be used to engineer cell lines which express the differentially expressed gene protein. Such engineered cell lines may be particularly useful in screening and evaluation of compounds that affect the endogenous activity of the differentially expressed gene protein.

When used as a component in assay systems such as those described below, the differentially expressed gene protein may be labeled, either directly or indirectly, to facilitate detection of a complex formed between the differentially expressed gene protein and a test substance. Any of a variety of suitable labeling systems may be used including but not limited to radioisotopes such as ¹²⁵I; enzyme labeling systems that generate a detectable calorimetric signal or light when exposed to substrate; and fluorescent labels.

Where recombinant DNA technology is used to produce the differentially expressed gene protein for such assay systems, it may be advantageous to engineer fusion proteins that can facilitate labeling, immobilization and/or detection.

Indirect labeling involves the use of a protein, such as a labeled antibody, which specifically binds to either a differentially expressed gene product.

Described herein are methods for the production of antibodies capable of specifically recognizing one or more differentially expressed gene epitopes. Such antibodies may include, but are not limited to polyclonal antibodies, monoclonal antibodies (mAbs), humanized or chimeric antibodies, single chain antibodies, Fab fragments, F(ab′)₂ fragments, fragments produced by a Fab expression library, anti-idiotypic (anti-Id) antibodies, and epitope-binding fragments of any of the above. Such antibodies may be used, for example, in the detection of a fingerprint, target gene in a biological sample, or, alternatively, as a method for the inhibition of abnormal target gene activity. Thus, such antibodies may be utilized for regeneration and sprouting and functional recovery of the nervous system.

For the production of antibodies to a differentially expressed gene, various host animals may be immunized by injection with a differentially expressed gene protein, or a portion thereof. Such host animals may include but are not limited to rabbits, mice, and rats, to name but a few. Various adjuvants may be used to increase the immunological response, depending on the host species, including but not limited to Freund's (complete and incomplete), mineral gels such as aluminum hydroxide, surface active substances such as lysolecithin, pluronic polyols, polyanions, peptides, oil emulsions, keyhole limpet hemocyanin, dinitrophenol, and potentially useful human adjuvants such as BCG (bacille Calmette-Guerin) and Corynebacterium parvum.

Polyclonal antibodies are heterogeneous populations of antibody molecules derived from the sera of animals immunized with an antigen, such as target gene product, or an antigenic functional derivative thereof. For the production of polyclonal antibodies, host animals such as those described above, may be immunized by injection with differentially expressed gene product supplemented with adjuvants as also described above. Monoclonal antibodies, which are homogeneous populations of antibodies to a particular antigen, may be obtained by any technique which provides for the production of antibody molecules by continuous cell lines in culture. These include, but are not limited to the hybridoma technique of Kohler and Milstein, (e.g. U.S. Pat. No. 4,376,110), the human B-cell hybridoma technique (e.g. Cole et al., 1983, Proc. Natl. Acad. Sci. USA 80:2026-2030), and the EBV-hybridoma technique (e.g. Cole et al., 1985, Monoclonal Antibodies And Cancer Therapy, Alan R. Liss, Inc., pp. 77-96). Such antibodies may be of any immunoglobulin class including IgG, IgM, IgE, IgA, IgD and any subclass thereof. The hybridoma producing the mAb of this invention may be cultivated in vitro or in vivo. Production of high titers of mAbs in vivo makes this the presently preferred method of production.

In addition, techniques developed for the production of “chimeric antibodies” (e.g. Morrison et al., 1984, Proc. Natl. Acad. Sci., 81:6851-6855) by splicing the genes from a mouse antibody molecule of appropriate antigen specificity together with genes from a human antibody molecule of appropriate biological activity can be used. A chimeric antibody is a molecule in which different portions are derived from different animal species, such as those having a variable or hypervariable region derived from a murine mAb and a human immunoglobulin constant region.

Alternatively, techniques described for the production of single chain antibodies (e.g. U.S. Pat. No. 4,946,778) can be adapted to produce differentially expressed gene-single chain antibodies. Single chain antibodies are formed by linking the heavy and light chain fragments of the Fv region via an amino acid bridge, resulting in a single chain polypeptide. Most preferably, techniques useful for the production of “humanized antibodies” can be adapted to produce antibodies to the polypeptides, fragments, derivatives, and functional equivalents disclosed herein. Such techniques are disclosed e.g. In U.S. Pat. No. 5,770,429, the disclosures of which are incorporated by reference herein in their entirety. Antibody fragments which recognize specific epitopes may be generated by known techniques.

Preferred antibodies of the present invention are expected to have, inter alla, similar biological functions/properties to a previously disclosed antibody (=IN-1) for the ligand of NgRH1) (see e.g. Schnell and Schwab, 1990, Nature 343, 269-272). Furthermore, preferred polypeptides and polynucleotides of the present invention have at least one activity of IN-1. An array of oligonucleotides probes comprising the GBRS polynucleotide sequence or fragments thereof can be constructed to conduct efficient screening of e.g., genetic mutations. Such arrays are preferably high density arrays or grids. Array technology methods are well known and have general applicability and can be used to address a variety of questions in molecular genetics including gene expression, genetic linkage, and genetic variability, see, for example, M. Chee et al., Science, 274, 610-613 (1996) and other references cited therein.

Detection of abnormally decreased or increased levels of polypeptide or mRNA expression may also be used for diagnosing or determining susceptibility of a subject to a disease of the invention. Decreased or increased expression can be measured at the RNA level using any of the methods well known in the art for the quantitation of polynucleotides, such as, for example, nucleic acid amplification, for instance PCR, RT-PCR, RNase protection, Northern blotting and other hybridization methods. Assay techniques that can be used to determine levels of a protein, such as a polypeptide of the present invention, in a sample derived from a host are well-known to those skilled in the art. Such assay methods include radioimmunoassays, competitive-binding assays, Western Blot analysis and ELISA assays.

Thus in another aspect, the present invention relates to a diagnostic kit comprising:

(a) a polynucleotide of the present invention, preferably the nucleotide sequence of SEQ ID NO: 1 or SEQ ID NO: 24, or a fragment or an RNA transcript thereof;

(b) a nucleotide sequence complementary to that of (a);

(c) a polypeptide of the present invention, preferably the polypeptide of SEQ ID NO: 2 or SEQ ID NO: 25 or a fragment thereof; or

(d) an antibody to a polypeptide of the present invention, preferably to the polypeptide of SEQ ID NO: 2.

It will be appreciated that in any such kit, (a), (b), (c) or (d) may comprise a substantial component. Such a kit will be of use in diagnosing a disease or susceptibility to a disease, particularly diseases of the invention, amongst others.

A further embodiment of the invention relates to methods to identify compounds that stimulate or inhibit the function or level of the polypeptide. Accordingly, in a further aspect, the present invention provides for a method of screening compounds to identify those that stimulate or inhibit the function or level of the polypeptide (e.g. blocking or stimulating NIH 3T3 cell spreading in vitro, blocking and stimulating PC12 neurite growth, inducing or blocking dorsal root ganglia growth cone collapse, spreading or blocking of neural cells, regeneration of lesioned nerve fibers in in vivo models). Such methods identify agonists or antagonists that may be employed for therapeutic and prophylactic purposes for such diseases of the invention as hereinbefore mentioned. Compounds may be identified from a variety of sources, for example, cells, cell-free preparations, chemical libraries, collections of chemical compounds, and natural product mixtures. Such agonists or antagonists so-identified may be natural or modified substrates, new ligands etc., as the case may be, of the polypeptide; a structural or functional mimetic thereof (see Coligan et al., Current Protocols in Immunology 1(2): Chapter 5 (1991)) or a small molecule.

The method may simply be a method of identifying a compound that modulates NgRH1 receptor activity, comprising:

(a) combining the compound with a NgRH1 receptor, preferentially human NgRH1, most preferentially a receptor comprising the amino add sequence as set forth in SEQ ID NO:2 or SEQ ID NO: 25; and

(b) measuring an effect of the compound on the receptor.

The screening method may simply measure the binding of a candidate compound to the polypeptide, or to cells or membranes bearing the polypeptide, or a fusion protein thereof, by means of a label directly or indirectly associated with the candidate compound. Alternatively, the screening method may involve measuring or detecting (qualitatively or quantitatively) the competitive binding of a candidate compound to the polypeptide against a labelled competitor (e.g. agonist or antagonist). Further, these screening methods may test whether the candidate compound results in a signal generated by activation or inhibition of the polypeptide, using detection systems appropriate to the cells bearing the polypeptide. inhibitors of activation are generally assayed in the presence of a known agonist and the effect on activation by the agonist by the presence of the candidate compound is observed. Further, the screening methods may simply comprise the steps of mixing a candidate compound with a solution containing a polypeptide of the present invention, to form a mixture, measuring a NgRH1 binding or activity in the mixture, and comparing the NgRH1 binding or activity of the mixture to a control mixture which contains no candidate compound. Polypeptides of the present invention may be employed in conventional low capacity screening methods and also in high-throughput screening (HTS) formats. Such HTS formats include not only the well-established use of 96- and, more recently, 384-well micotiter plates but also emerging methods such as the nanowell method described by Schullek et al, Anal Biochem., 246, 20-29, (1997).

The polynucleotides, polypeptides and antibodies to the polypeptide of the present invention may also be used to configure screening methods for detecting the effect of added compounds on the production of mRNA and polypeptide in cells. For example, an ELISA assay may be constructed for measuring secreted or cell associated levels of polypeptide using monoclonal and polyclonal antibodies by standard methods known in the art. This can be used to discover agents that may inhibit or enhance the production of polypeptide (also called antagonist or agonist, respectively) from suitably manipulated cells or tissues.

An additional embodiment of the invention relates to the administration of a pharmaceutical composition, in conjunction with a pharmaceutically acceptable carrier, for any of the therapeutic effects discussed above. Such pharmaceutical compositions may consist of antibodies to NgRH1s, mimetics, agonists, antagonists, or inhibitors of NgRH1s. The compositions may be administered alone or in combination with at least one other agent, such as stabilizing compound, which may be administered in any sterile, biocompatible pharmaceutical carrier, including, but not limited to, saline, buffered saline, dextrose, and water. The compositions may be administered to a patient alone, or in combination with other agents, drugs or hormones.

The pharmaceutical compositions encompassed by the invention may be administered by any number of routes including, but not limited to, oral, intravenous, intramuscular, intra-articular, intra-arterial, intramedullary, intrathecal, intraventricular, transdermal, subcutaneous, intraperitoneal, intranasal, enteral, topical, sublingual, or rectal means.

In addition to the active ingredients, these pharmaceutical compositions may contain suitable pharmaceutically-acceptable carriers comprising excipients and auxiliaries which facilitate processing of the active compounds into preparations which can be used pharmaceutically. Further details on techniques for formulation and administration may be found in the latest edition of Remington's Pharmaceutical Sciences (Maack Publishing Co., Easton, Pa.).

The pharmaceutical composition may be provided as a salt and can be formed with many acids, including but not limited to, hydrochloric, sulfuric, acetic, lactic, tartaric, malic, succinic, etc. Salts tend to be more soluble in aqueous or other protonic solvents than are the corresponding free base forms. In other cases, the preferred preparation may be a lyophilized powder which may contain any or all of the following: 1-50 mM histidine, 0.1%-2% sucrose, and 2-7% mannitol, at a pH range of 4.5 to 5.5, that is combined with buffer prior to use.

For any compound, the therapeutically effective dose can be estimated initially either in cell culture assays, e.g., of neoplastic cells, or in animal models, usually mice, rabbits, dogs, or pigs. The animal model may also be used to determine the appropriate concentration range and route of administration. Such information can then be used to determine useful doses and routes for administration in humans. A therapeutically effective dose refers to that amount of active ingredient, for example antibodies, agonists, antagonists or inhibitors of NgRH1, which ameliorates the symptoms or condition. Therapeutic efficacy and toxicity may be determined by standard pharmaceutical procedures in cell cultures or experimental animals, e.g., ED50 (the dose therapeutically effective in 50% of the population) and LD50 (the dose lethal to 50% of the population). The dose ratio between toxic and therapeutic effects is the therapeutic index, and it can be expressed as the ratio, LD50/ED50. Pharmaceutical compositions that exhibit large therapeutic indices are preferred. The data obtained from cell culture assays and animal studies is used in formulating a range of dosage for human use. The dosage contained in such compositions is preferably within a range of circulating concentrations that include the ED50 with little or no toxicity. The dosage varies within this range depending upon the dosage form employed, sensitivity of the patient, and the route of administration.

The exact dosage will be determined by the practitioner, in light of factors related to the subject that requires treatment. Dosage and administration are adjusted to provide sufficient levels of the active moiety or to maintain the desired effect. Factors that may be taken into account include the severity of the disease state, general health of the subject, age, weight, and gender of the subject, diet, time and frequency of administration, drug combination(s), reaction sensitivities, and tolerance/response to therapy. Long-acting pharmaceutical compositions may be administered every 3 to 4 days, every week, or once every two weeks depending on half-life and clearance rate of the particular formulation.

Normal dosage amounts may vary from 0.1 to 100,000 micrograms, up to a total dose of about 1 g, depending upon the route of administration. Guidance as to particular dosages and methods of delivery is provided in the literature and generally available to practitioners in the art. Those skilled in the art will employ different formulations for nucleotides than for proteins or their inhibitors. Similarly, delivery of polynucleotides or polypeptides will be specific to particular cells, conditions, locations, etc. Pharmaceutical formulations suitable for oral administration of proteins are described, e.g., In U.S. Pat. Nos. 5,008,114; 5,505,962; 5,641,515; 5,681,811; 5,700,486; 5,766,633; 5,792,451; 5,853,748; 5,972,387; 5,976,569; and 6,051,561.

The following Examples illustrate the present invention, without in any way limiting the scope thereof.

### EXAMPLES

### Example 1

### Isolation of the Human NgRH1 cDNA

Identification of human NgRH1 and sequence analysis—Using the published NgR amino acid sequence (accession number: AF283463) as a query, tblastn search brought up two sequences from the Celera database, hCT31020 and GA_{—}53256868, the translational products of which showed roughly 50 percent similarity (blast result) to the published NgR sequence. Alignments were performed, using either GAP or BESTFIT analysis from the Wisconsin Package Version 10.1, Genetics Computer Group (GCG), Madison, Wis. Multiple sequence analysis were done by ClustalW. Signal peptide prediction for NgR and the homologues were done using Spscan. Leucine-rich-repeats (LRR) were identified through comparing sequences with various consensus segments present in the protein-family-database Pfam. Further identification and confirmation of LRR's were done by BLOCKS+ alignments using the IMPALA software. Prediction of a potential GPI cleavage site was done using DGPI, from the ExPaSy program package present on the server from the Swiss Institute of Bioinformatics (SIB).

Isolation of full length cDNAs encoding human NgRH1: The cDNA for the NgR homologue-1 was obtained by PCR from a human brain cDNA (Marathon-Ready™ cDNA, CLONTECH Laboratories, Inc., Palo Alto, Calif., cat. Nr. 7400-1). The 5′- and the 3′ end of the gene were amplified separately (see below). Sequence specific primers were designed based on a continuous sequence assembled from a predicted transcript sequence from the Celera database (accession number hCT31020) and two EST sequences from public DNA data bases (accession numbers: A1929019 and BE410139). PCRs were carried out on a PerkinElmer GeneAmp 9600 cycler, using 4% DMSO and Herculase™ Enhanced DNA-Polymerase (Stratagene Europe, Amsterdam, Netherlands). The 5′ end (798 bp) was amplified from total brain Marathon-Ready™ cDNA (Clontech Laboratories, Inc., Palo Alto, Calif., cat. Nr. 7400-1) by 5′-RACE. A first amplification was carried out using the AP1 primer from the kit and a sequence specific primer GSP1 (5′-GTG GTT GGA GGA GGC CTG GM GT-3′ (SEQ ID NO: 3)). Nested PCR using the AP2 primer from the kit and sequence specific primer nGSP1 (5′-GGC CAG GCT GTT GTT GAA CAG G-3′ (SEQ ID NO: 4)) resulted in a 798 bp fragment which was ligated into the pCR-Blunt 1′-TOPO cloning vector (Invitrogen, Basel, Switzerland). The correct sequence was confirmed by automated sequence analysis. The 3′ end of the gene (804 bp) was amplified from a human fetal brain cDNA (Marathon Ready, Clontech Laboratories, Inc., Palo Alto, Calif., cat. Nr. 7402-1) by PCR using gene specific primers GSP2 (5′-GGT CAG CCT GCA GTA CCT CTA CC-3′ (SEQ ID NO: 5)) and NgRH1-3′ (5′-AGT CAG AGG TGG TGG GGC ACC AG-3′ (SEQ ID NO: 6)). The PCR fragment was ligated into pCR TOPO TA-vector (Invitrogen, Basel, Switzerland) and sequenced. The full length cDNA was then assembled by overlapping PCR using the primers NgRH1-5′ (5′-AGA TGC TGC CCG GGC TCA GGC G-3′ (SEQ ID NO: 7)) and NgRH1-3′ (see above). The PCR product was ligated into pCR-Blunt II-TOPO cloning vector. This final plasmid is called hNgRH1-fl hereafter. Sequence analysis confirmed the predicted sequence from the Celera database except a point mutation (silent) at position 642 (G+A) and an additional 51 bases inserted at position 926 of the predicted sequence. The 51 bp insert was confirmed by the presence of another EST in Genbank (accession number BE222737). Recently a sequence has been deposited in the GenBank database (accession number AX411529) that matches to our NgRH1 sequence. The human NgRH1 is 45% similar on the amino acid level to human NgR.

Not only the complete DNA-sequence for human NgRH1 (SEQ ID NO: 1) can be obtained with this approach (1263 bp coding for 420 amino acids with a molecular weight of 42 kDa), but also the disclosed other variants and fragments of the invention. As an alternative, the first step in obtaining NgRH1s may start with using the amino acid sequence of human NgRH1.

Leucine-rich-repeats (LRR) are identified through comparing sequences with various consensus segments present in the protein-family-database Pfam. Further identification and confirmation of LRR's are done by BLOCKS+ alignments using the IMPALA software. Prediction of a potential GPI cleavage site is done using DGPI, from the ExPaSy program package present on the server from the Swiss Institute of Bioinformatics (SIB).

Thus, Human NgRH1 appears to belong to the same family of leucine-rich/proteoglycan proteins as NgR. Like human NgR, human NgRH1 codes for also 8 LRR's flanked by a leucine-rich-repeat-N-terminus and a leucine-rich-repeat-C-terminus. In addition to the presence of a signal sequence at the N-terminus, human NgRH1 contains a short hydrophobic amino acid stretch at its C-terminus, typical for GPI-linked proteins (see also Example 3).

The rat gene can be obtained by an analogous method. The cDNA coding for rat-NgRH1 is amplified by PCR from a rat brain cDNA-library (Marathon-Ready cDNA, BD Clontech, Palo Alto). PCR is performed according to standard protocols using Herculase Enhanced DNA Polymerase (Stratagene Europe, Amsterdam, Netherlands). Primers are chosen based on the sequence of the 5′-UTR of human NgRH1 (SEQ ID NO:1) (5′-TGAATCTGGACCCCGGGAGG-3′ (SEQ ID NO: 8)) and the rat EST-sequence, acc. # BE097332 (5′-TCCTCAGCGGAGAGATACCACCA-3′ (SEQ ID NO: 9)). The full-length cDNA was cloned into pCR-TOPO-Blunt (Invitrogen). The full-length cDNA for rat-NgRH1 with the human sequence is 87% Identical on DNA level and 88% on amino acid level.

### Example 2

### Human NgR and NgRH1 Expression and their Biochemical Characterization

Materials—Media: MEM-alpha-plus medium and OptiMEM I with Glutamax (Invitrogen, Basel, Switzerland). Primary antibodies: monoclonal anti-V5 antibody (Invitrogen, Basel, Switzerland); monoclonal anti-HA antibody, clone HA-7 (SIGMA, Buchs, Switzerland). Secondary antibodies: POD conjugated anti-mouse or anti-rabbit IgG antibody (SIGMA, Buchs, Switzerland); Anti-mouse IgG, FITC conjugated antibody (SIGMA, Buchs, Switzerland). Complete™ (Roche Applied Science (Rotkreuz, Switzerland).

Methods:

a) Northernblots: In order to determine the tissue distribution of hNgR and hNgRH1 expression, a Multiple Tissue Northernblot (MTN, CLONTECH Laboratories, Inc., Palo Alto, Calif.) and a Multiple Tissue Expression Array (MTE array, CLONTECH Laboratories, Inc., Palo Alto, Calif.) was hybridized with α-³²P-dCTP and α-³²P-dATP-radiolabeled human NgR and NgRH1 probe. The probes for the respective cDNAs were generated as follows. NgR probe was generated by excision of pcDNA-Sport6-NgR by EcoRI/Xho-I cleavage. This clone was obtained from a human dorsal root ganglion (DRG) cDNA library (Life Technologies Inc., Rockville, Md.). It was identified through a blast search against library clone sequences, using the human NgR cDNA as a query. The cDNA insert of pcDNA-Sport6-NgR is 24 and 292 bp longer on the 5′end and 3′ end respectively, compared to the published sequence for NgR (accession number. AF283463). The NgRH1 probe was generated by excision of hNgRH1-fl by EcoRI cleavage. The resulting 1.8 kb and 1.3 kb cDNA inserts for NgR and NgRH1 respectively were gel purified (QIAEX II Gel Extraction Kit, QIAGEN AG, Basel, Switzerland) and 100 ng each was radiolabeled using High Prime DNA Labelling Kit (Roche Biochemicals, Rothkreuz, Switzerland) In three separate labelling reactions. Unincorporated nucleotides were removed, using the NucTrap Probe Purification Columns (Stratagene Europe, Amsterdam, Netherlands). MTN or MITE membranes were hybridised with either NgR or NgRH1 probe in ExpressHyb solution (CLONTECH Laboratories, Inc., Palo Alto, Calif.) according to the manufacturer instructions (except that the Cot-1 DNA was left out). A single band at 2.4 kb for human NgR and human NgRH1 is detected. Highest mRNA expression of human NgR and human NgRH1 is in the human brain. However, abundant expression of human HgRH1 can be seen in human liver too. Both genes are expressed at low levels in other peripheral tissues, such as skeletal muscle, spleen, kidney, lung and placenta. In order to get a more detailed view of potential spatial differences in brain expression, we also probed MTE's, carrying spotted RNAs from various brain regions. From this analysis, it appeared that both genes were differentially expressed in different brain areas. While they are strongly expressed in the cerebral cortex, amygdala, hippocampus and accumbens nucleus, only NgR is highly abundant in the cerebellum, compared to expression in the cortex. Common to both genes is their weak expression in pons, corpus callosum, caudata nucleus, medulla oblongata, putamen, substancia nigra and spinal cord.

b) Cell culture and transfection: CHO-K1 cells are grown in MEM-alpha-plus medium. This medium is supplemented with 10% Fetal Calf Serum (FCS) final concentration and Penicillin Streptomycin to a final concentration of 200 U/ml. For transient and stable cell transfection, FUGENE 6 (Roche Applied Science, Rotkreuz, Switzerland) is used according to the manufacture instructions. Cells expressing human NgR and human NgRH1 (pSecTag2B vector) were put under selection with Zeocin to a 0.25 mg/ml final concentration.

c) Construction of epitope tagged NgR and NgRH1-1.) NgR-V5 tag cloning procedure: Two complementary, synthetic oligonucleotides (Microsynth, Balgach, Switzerland) 5′-CCG GTA AGC CTA TCC CTA ACC CTC TCC TCG GTC TCG ATT CTA CGT CTA GAT ATC CTC GAG-3′ (SEQ ID NO: 10) and 5′-GAG CTC CTA TAG ATC TGC ATC TTA GCT. CTG GCT CCT CTC CCA ATC CCT ATC CGA ATG GCC CGA-3′ (SEQ ID NO: 11), coding for the V5-tag and restriction cleavage sites XbaI/EcoRV/XhoI were annealed and ligated into the SfiI-PmeI sites of pSecTag2B vector (INVITROGEN, Basel, Switzerland) to get pSecTag2-V5. After this, the cDNA sequence coding for human NgR, without the signal peptide, was amplified by PCR from pcDNA-Sport8NgR (see above) using forward primer 5′-GCA GCA TCT AGA CCA GGT GCC TGC GTA TGC TAC AAT GAG CCC-3′ (SEQ ID NO: 12) and reverse primer 5′-GCA GCA CTC GAG TCA GCA GGG CCC AAG CAC TGT CCA CAG CAC-3′ (SEQ ID NO: 13), cleaved with XbaI and XhoI and ligated into the respective cleavage sites in pSecTag2-V5. 2.) NgRH1-HA tag cloning procedure: Two complementary, synthetic oligonucleotides (Microsynth, Balgach, Switzerland) 5′CCG ATT ACA AGG ATG ACG ACG ATA AGT CTA GAC AGT GCG ATA TCA ATG AAT TC-3′ (SEQ ID NO: 14) and 5° CTT AAG TAA CTA TAG CGT GAC AGA TCT GAA TAG CAG CAG TAG GAA CAT TAG CCC GA-3′ (SEQ ID NO: 15), coding for the FLAG-tag and restriction cleavage sites XbaI/EcoRV/EcoRI were annealed and ligated into the SfiI-PmeI sites of pSecTag2B vector (INVITROGEN, Basel, Switzerland) to get pSecTag2-FLAG. After that, the cDNA sequence coding for human NgRH1, without the signal peptide, was amplified from hNgRH1-fl by PCR using the forward primer 5′-MT TGC GCA TCT AGA GCC CCC AGC TGC CCC ATG CTC TGC ACC TGC3′ (SEQ ID NO: 16) and reverse primer 5′-AAT TGC GCA GAA TTC TCA GAG GTG GTG GGG CAC CAG CAG CAG GAG-3′ (SEQ ID NO: 17), cleaved with XbaI and EcoRI and ligated into the respective cleavage sites in pSecTag2-FLAG.

Following stable cell transfection into CHO-K1 cells, human NgR and NgRH1 tagged with V5- or HA-tags respectively, were analysed in Westernblots. The respective cell lines expressed proteins larger than the molecular weights predicted for NgR (47 kDa) and NgRH1 (42 kDa), at around 64 kDa. As we show later, the aberrant molecular weights can be explained by post-translational modification. At least two major forms for NgR are produced by the CHO cells. A protein band seen at 64 kDa, most likely corresponds to the full length NgR. The other one, at approximately 48 kDa, seems to be a truncated NgR molecule. The 48 kDa band however wasn't detectable with the polyclonal α-NgR antisera. This is either due to the unspecific band running close to it, that makes the identification impossible, or this form of NgR lacks epitopes against which the antisera was raised. In contrast, the individual protein bands for NgRH1 could be confirmed with the specific polyclonal antisera. With the exception of α-NgR, none of the anti-sera picked up proteins in the untransfected control cells, nor did they cross-react to different NgR species.

d) Cell surface expression of NgR and NgRH1-1.) Antibodies and Immunological detections—For the detection of NgR and NgRH1 proteins, either commercially available monoclonal anti-tag antibodies or polyclonal antisera, raised in rabbits, were used. Generation of polyclonal antisera: Rabbit anti-NgR antisera was obtained form M. Schwab, University of Zürich raised against three synthetic peptides from human NgR (EQLDLSDNAQLRSVDPA (SEQ ID NO: 18), EVPCSLPQRLAGRDLKR (SEQ ID NO: 19) and GPRRRPGCSRKNRTRS (SEQ ID NO: 20)) and affinity purified by Research Genetics (Invitrogen, Corporation). Rabbit NgRH1 antisera are raised against synthetic peptides (CPPAAPTRPGSRARGN (SEQ ID NO: 21), DLPAEDSRGRQGGDAP (SEQ ID NO: 22) and TEDDYWGGYGGEDQR (SEQ ID NO: 23)) derived from the human NgRH1 sequence and affinity purified by EUROGENTECS (Seraing, Belgium). 2.) Westernblots: SDS-gel electrophoresis were done using NuPAGE precast 4-12% Bis-Tris gels (INVITROGEN, Basel, Switzerland). Usually, MOPS running was used for the separation. Electroblotting of the gels was done using a Semiphor Transphor Unit (Amersham Biosciences Dübendorff, Switzerland), applying 24 V for 1 h. For immunodetection, the PVDF membranes were blocked for 45 min in 5% skimmed milk in TBST, followed by a one hour incubation with the primary antibody and secondary anti-mouse or anti-rabbit IgG antibody respectively, diluted in blocking solution. Membranes were washed three times after each antibody incubation in TBST, containing 10 mM Tris pH 7.5, 140 mM NaCl and 0.2% Tween 20, followed by a single wash in TBS. Signals were developed using ECL™ Western Blotting Detection Reagents (Amersham Biosciences, Dübendorf, Switzerland) and Hyperfilm™ ECL™ (Amersham Biosciences, Dibendorf, Switzerland) according to the manufacturer's instructions. 3.) Cell surface biotinylation: Cells at confluency were incubated for 30 minutes at 4° C. with PBS containing 1 mg/ml biotin 3-sulfo-N-hydroxysuccimide ester (Sulfo-NHS Biotin), rinsed three times with PBS containing 50 mM glycine and lysed in M-PER. Clarified lysates were incubated for 2 hr at room temperature with 20 μg/ml streptavidin coupled to agarose beads and the beads subsequently washed successively with 10 mM Tris-HCl pH 7.8, 1% (w/v) N-lauroylsarcosine, 100 mM NaCl prior to SDS-PAGE analysis. NgR and NgRH1 are readely biotinylated on the cell surface of stable CHO-K1 transfectants, whereas the control protein (GAPDH) is not. 4.) Immunoflourescence: For surface versus cytoplasmic staining, cells were either directly incubated with the primary antibody and then post-fixed, or cells were fixed in 4% paraformaldehyde and blocked in 10% FCS, 0.1% Triton X-100 for 20 minutes, prior to the primary antibody was added. A FITC coupled anti-mouse IgG antibody was used as secondary antibody. Pre-immune sera were used as controls and did not reveal unspecific background staining of intact cells.

In order to characterize the subcellular distribution of NgR and NgRH1 and to show that all three proteins are cell surface expressed, we performed cell surface biotinylation. Whereas no labeling of cytoplasmic control protein GAPDH was seen, NgR and NgRH1 molecules were readily biotinylated with the non-penetrable reagent Sulfo-NHS-Biotin, added to the cells. The cell surface expression of NgR and NgRH1 was confirmed by immunofluorescence of non-permeabelized, stable CHO cells, using either anti-tag antibodies or polyclonal antisera against the individual NgR proteins.

e) GPI-linkage and glycosylation of NgR and NgRH1: In the next couple of experiments we checked for post-translational modifications in vitro. Since a GPI-linkage of NgRH1 is predicted from their primary structures, we first checked this experimentally using PI-PLC. First, total cell extracts were incubated in the presence or absence of bacterial Phosphatidyl-Inositol-Phospholipase-C (PI-PLC). The removal of a GPI-linker alters the mobility of proteins in the SDS-PAGE (Cardoso de Almeida et al., (1983) Nature 302, 349-52; Stahl, N. et al, (1987) Cell 51, 229-40; Littlewood, G. M. et al., (1989) Biochem. J. 257, 361-7). Proteins with a GPI-linker usually get a higher loading with SDS molecules due to the hydrophobic interaction with the lipid chains of the GPI-linker. The net charge therefore becomes slightly more negative compared to proteins from which the GPI-linker has been enzymatically removed. Thus GPI-removal results in a characteristic up-shift of the respective protein in the SDS-PAGE, compared to untreated control proteins that are run in parallel lanes. To make the interpretation easier, cells were pretreated with Tunicamycin to reduce the protein complexity caused by glycosylation. Following this protocol, NgR, as well as NgRH1 indeed showed the characteristic up-shift in the SDS-PAGE after PI-PLC treatment, demonstrating that they are GPI-linked. The secreted form of NgR remains completely unaffected from the PI-PLC treatment. To further confirm these findings, cultured CHO cells expressing NgR or NgRH1 were incubated with PI-PLC. Subsequently, the proteins collected from the conditioned medium were analyzed by Westernblots, versus proteins remaining on the cells. NgR and NgRH1 were released into the conditioned medium after 3 h PI-PLC treatment, confirming that all of them are GPI-anchored. Interestingly, even in the absence of PI-PLC, there is considerable, constitutive secretion of all three different proteins. This is most apparent in case of the NgR expressing cell line, from which the truncated form of NgR (48 kDa) that was observed before, Is substantially released into the medium. Smaller proteins, but also potentially full length species of NgRH1 are detectable in the control conditioned medium as well. To rule out that these observations are specific to CHO cells, we confirmed the existence of two major forms for NgR transiently transfected COS-7 cells. There are bands seen in the SDS-PAGE emerging in the cell pellets of NgR and NgRH1 CHO cells, whose identification cannot be determined easily. A simple explanation would be that they stem from incompletely glycosylated NgR molecules. All NgR proteins contain putative N-glycosylation sites (Asn-X-Ser/Thr) in their amino acid sequence. Incubating the cells with Tunicamycin, markedly reduced the molecular weights of bands specific for NgR and NgRH1, demonstrating that all NgR proteins become highly glycosylated. The molecular weights now match nicely the predicted sizes for NgR and NgRH1, reflecting the unmodified proteins. For NgR, the smaller secreted form is still detectable, showing that this molecule is produced independently of glycosylation. Interestingly, whereas Tunicamycin readily prevented the release of NgR into the medium, it was less effective in case of NgRH1. Relatively high amounts of NgRH1 are still released into the medium after PI-PLC treatment, indicative of a longer half-life of this protein, compared to NgR. The Tunicamycin experiment clearly showed that NgR and NgRH1 are glycosylated and therefore we suggest that additional bands that cannot be assigned unambiguously to secreted or mature molecules in the cell pellet fraction, stem from immature precursors that have not undergone full post-translational modification. Interestingly, these immature forms only show up if the cells were PI-PLC treated. This might reflect increased de novo protein synthesis after removal of NgR proteins from the plasma membrane, as compared to steady state conditions.

f) Lipid raft association of NgR and NgRH1-1.) Phosphatidylinositol-specific phospholipase C (PIPLC) treatment: i) Treatment of intact cells: Cells at confluency were washed twice in OptiMEM and then incubated for 4 hrs at 37° C. In 4 ml OptiMEM containing 0.2 U/ml PI-PLC (GLYKO Inc., Novato, Calif.). After 5 minutes centrifugation at 3000 rpm, the cell medium was concentrated six times in a Centricon YM-10 (Millipore, Volketsawil, Switzerland). The remaining cells were washed twice with PBS and harvested with a cell scraper and centrifuged for 1 min at 5000 rpm. Cell lysis Cell pellets were lysed in M-PER (100 μl per 25 mg cell pellet), sublemented with Complete™ (ROCHE Applied Science, Rothkreuz, Switzerland) for 20 minutes at room temperature. After this, the lysed material was centrifuged for 10 minutes at 14000 rpm; 4° C. and sample buffer was added to supernatant. Equivalent volumes of lysate and concentrated medium were subjected to SDS-PAGE (Invitrogen 4-12% Bistris Gels, MOPS buffer) and blotted onto PVDF membranes. PI-PLC readily releases NgR and NgRH1 from transfected CHO-K1 and 293T cells into the conditioned medium after PI-PLC treatment, showing that they possess a GPI-anchor. ii) Treatment of cell lysates: Cells were grown to 50%-80% confluency. The medium was discarded and the cells were incubated at 37°; 5% CO₂ overnight in 3 ml OptiMEM (INVITROGEN, Basel, Switzerland) containing 5 μg Tunicamycin (GLYKO Inc., Novato, Calif.) per ml medium. The cells were then washed with PBS and harvested by scraping. The harvested cells were centrifuged for 1 min at 4° C. at 20000×g and lysed for 20 minutes at room temperature, in 200 μM-PER/Complete with EDTA per 100 mg of cell pellet. The sample was again centrifuged for 10 minutes at 4° C. with 20000×g and the supernatant was collected. This material was splited into two equal aliquots and diluted 1:2 with 10 mM Hepes pH 7.6 buffer. To one of the aliquots 1 U/ml PI-PLC was added and both aliquots were incubate for at least 3 h at 37° C. Following PI-PLC treatment of cell lysates, NgR and NgRH1 show a characteristic upshift on SDS-PAGE indicative of the removal of the GPI anchor (Cardoso et al. (1983) Nature 302, 349-52; Stahl et al. (1987) Cell 51, 22940; Uttlewood et al. (1989) Biochem. J. 257, 361-7). Lipid raft isolation: Lipid raft preparation was carried out after Brown and Rose 1992 (Brown et al. (1983) Nature 302, 349-52). Briefly, cells at confluency from a 10 cm dish were washed with MBS (25 mM MES-buffer/0.15M NaCl, pH 6.5) and scraped into same buffer. After centrifugation at 1200 rpm for 5 minutes the cells were resuspended on ice in 0.3 ml MBS. All following steps were carried out at 4° C. The sample was adjusted to approx. 0.5 ml with MBS and to a final concentration of 1% Triton X-100 and homogenised in a Dounce homogeniser. Homogenised samples were mixed with equal volume of 80% (w/v) sucrose/MBS (0.5 ml) for final concentration of 40% (w/v) sucrose. The samples were then filled under the 5%/30% layers of sucrose gradients. Centrifugation of the gradients were carried out at 37 200 rpm in SW50.1 rotor for 16-18 hr at 4° C. Lipid rafts/DRMs appear at the 5%/30% Interface as an opaque band. 9×0.5 ml fractions were harvested from bottom to top of the gradient using a 1 ml syringe/21G needle.

GPI-linked proteins are a characteristic constituent of lipid rafts. These microdomains of the plasma membrane are functionally and biophysically distinct from the regular organization of the plasma membrane structure, known as the phospholipid biolayer. Asymmetrical packing of specific lipids, such as cholesterol and sphingolipids, determines a liquid-ordered state of rafts that result in an insolubility in Triton X-100, at low temperature. This behavior has led to the name detergent-resistant-membranes (DRM), which is used as a technical term, synonymous to raft or caveolae. DRM's float to a low density in sucrose gradients, due to their high lipid content. This enables any associated protein to be identified and distinguished from other soluble components of the cells. Therefore, we extracted CHO cells expressing NgR and NgRH1 in 1% Triton X-100 at 4° C. and the resulting extracts were applied to sucrose density centrifugation. Under our experimental set up, sedimentation of DRM's usually takes place at the 5%/30% sucrose interface (fraction 5 and 6) together with proteins known to be associated with rafts, such as Flotillin. NgR and NgRH1 proteins indeed sedimented in the expected fractions 5 and 6, together with the marker protein, demonstrating their lipid raft association. In contrast, the secreted form of NgR does not co-sediment with rafts, but smears through several fractions from bottom to top of the sucrose gradient, mainly residing in high density fractions at 40% sucrose, containing Triton X-100 soluble material. This is also true for the secreted forms from NgRH1 that are found mainly in fractions 1 and 2. Two protein bands for Flotillin are detectable in the sucrose gradient, one band at 48 kDa and a smaller one at approximately 45 kDa. Both were originally described by Bickel et al. (1997) J. Biol. Chem. 272, 13793-802). The lower band at 45 kDa was found to be cross-reacting with the antibody and is found in our hands in Triton X-100 soluble fractions of the sucrose gradient only.

Thus, we describe the characterization of proteins that were identified as homologues of the recently described receptor of Nogo-68. NgRH1 are highly related to NgR terms of primary structure, biochemical properties and expression pattern. Multiple lines of evidences as presented above support the conclusion that NgR and the newly identified homologue NgRH1 are members of a novel protein family.

### Example 3

### Ligand Binding Analysis

Ligand binding assays provide a direct method for ascertaining receptor pharmacology and are adaptable to a high throughput format. The purified ligand (putatively NogoA, NogoB, NogoC, Nogo-66, MAG of OMgp) for the receptor hNgRH1 may be radiolabeled to high specific activity (50-2000 Ci/mmol) for binding studies (or using suitable detection tags to the ligands (agonists or antagonists) such as, alkaline phosphatase, GST, Myc, His, V5 etc). A determination may be then made that the process of radiolabeling (or other signals) does not diminish the activity of the ligand towards its receptor. Assay conditions for buffers, ions, pH and other modulators such as nucleotides may be optimized to establish a workable signal to noise ratio for both membrane and whole cell receptor sources. For these assays, specific receptor binding may be defined as total associated radioactivity minus the radioactivity measured in the presence of an excess of unlabeled competing ligand or in the presence of an excess of the soluble NgRH1 ectodomain, lacking the GPI-anchor (Domeniconi et al. (2002) Neuron 35, 283-290 (published online June 28), Liu et al. (2002) Science June 27 (epub ahead of print). Where possible, more than one competing ligand may be used to define residual nonspecific binding.

### Example 4

### Functional Assays

Human NgRH1 may be expressed in recombinant expression systems such as HEK293 cells, CHO cells or COS cells and verified for expression at the cell surface (e.g. see Example 2d). Alternatively, hNgRH1 is expressed in recombinant expression systems as above together with putative interacting proteins (e.g. Nogo-66 or NogoA, NogoB, NogoC, MAG or OMgp). Co-transfection of cDNA expression constructs is for example done with the Effectene transfection agent (Qiagen). A functional read-out may involve analysis of agonist (e.g. by application or co-expression of Nogo-A,B,C, MAG or OMgp to CHO cells stably expressing NgRH1) induced change in cell adhesion, cell sprouting, intracellular CAMP levels and intracellular Ca²⁺ levels.

Alternatively, effect of compounds, antibodies and molecules blocking or down-regulating the receptors is assessed and confirmed in standard functional assays for growth cone collapse, neurite outgrowth and spreading of 3T3 cells in the presence of Nogo ligands (e.g. Nogo A or C) as described in the following papers (Chen et al., 2001, Nature 403, 434-439; Fournier et al., 2001, Nature 409, 341-346). Regenerative effects of these therapeutic agents is also be assessed in in vivo models of brain and spinal injury as described e.g. In the following paper (e.g. Schnell et al., 1990, Nature 343, 269-272) and effect on functional deficits (e.g. Thallmair et al., 1998, Nature Neurosci. 1, 124-131; Z'Graggen et al., 1998, J. Neurosci. 18, 4744-4754).

1. An isolated polypeptide selected from one of the groups consisting of:
(a) an isolated polypeptide encoded by a polynucleotide comprising the sequence of SEQ ID NO: 1 or SEQ ID NO: 24;
(b) an isolated polypeptide comprising a polypeptide sequence having at least 95% identity to the polypeptide sequence of SEQ ID NO: 2 or SEQ ID NO: 25;
(c) the polypeptide sequence of SEQ ID NO: 2 or SEQ ID NO: 25 and
(d) fragments of such polypeptides in (a) to (c).

2. The isolated polypeptide according to claim 1, wherein the polypeptide sequence is SEQ ID NO: 2 or SEQ ID NO: 25.

3. An isolated polynucleotide selected from one of the groups consisting of:
(a) an isolated polynucleotide comprising a polynucleotide sequence having at least 95% identity to the polynucleotide sequence of SEQ ID NO: 1 or SEQ ID NO: 24;
(b) an isolated polynucleotide comprising a polynucleotide sequence encoding a polypeptide sequence having at least 95% identity to the polypeptide sequence of SEQ ID NO: 2 or SEQ ID NO: 25;
(c) an isolated polynucleotide comprising a nucleotide sequence of at least 100 nucleotides;
(d) a polynucleotide which is the RNA equivalent of a polynucleotide of (a) to (d); or a polynucleotide sequence complementary to said isolated polynucleotide and polynucleotides that are variants and fragments of the above mentioned polynucleotides or that are complementary to above mentioned polynucleotides, over the entire length thereof.
(e) an isolated polynucleotide which hybridizes under stringent hybridization conditions to SEQ ID No. 1 or SEQ ID No. 24, complements or fragments thereof;

4. The isolated polynucleotide according to claim 3 wherein said polynucleotide sequence is 30 nucleotides, 30-50 nucleotides, 50-100 nucleotides or 100-200 nucleotides in length.

5. An expression vector comprising a polynucleotide capable of producing a polypeptide of claim 1 when said expression vector is present in a compatible host cell.

6. A recombinant host cell comprising the expression vector of claim 5.

7. A process for producing a polypeptide of claim 1 comprising the step of culturing a host cell under conditions sufficient for the production of said polypeptide and recovering the polypeptide from the culture medium.

8. A fusion protein comprising the Immunoglobulin Fc-region and a polypeptide of claim 1.

9. An antibody immunospecific for the polypeptide of claim 1.

10. A method for identifying a compound that modulates NgRH1 receptor activity, comprising:
(a) combining the compound with a polypeptide according to claim 1; and
(b) measuring an effect of the compound on the receptor.

11. The method according to claim 10, wherein said NgRH1 receptor is a human NgRH1 receptor comprising the amino acid sequence as set forth in SEQ ID NO: 2.

12. A purified antibody or a fragment thereof which specifically binds to a polypeptide that comprises the amino acid sequence set forth in SEQ ID NO: 2 or SEQ ID NO: 25 or a fragment of a polypeptide that comprises the amino acid sequence set forth in SEQ ID NO: 2 or SEQ ID NO: 25.

13. An antibody fragment according to claim 12 which is an Fab or F(ab′)₂ fragment.

14. The antibody according to claim 12, which is a polyclonal antibody.

15. The antibody according to claim 12, which
